(19) United States
(12) Patent Application Publication
(10) Pub. No.: US 2013/0090642 A1
(43) Pub. Date: Apr. 11, 2013

(54) LAPARSCOPIC TISSUE MORCELLATOR SYSTEMS AND METHODS

(71) Applicants: John H. Shadduck, Menlo Park, CA (US); Aaron Germain, Campbell, CA (US); Csaba Truckai, Saratoga, CA (US); Michael D. Walker, Mountain View, CA (US); Kyle Klein, San Jose, CA (US)

(72) Inventors: John H. Shadduck, Menlo Park, CA (US); Aaron Germain, Campbell, CA (US); Csaba Truckai, Saratoga, CA (US); Michael D. Walker, Mountain View, CA (US); Kyle Klein, San Jose, CA (US)

(73) Assignee: ARQOS Surgical, Inc., Cupertino, CA (US)

(21) Appl. No.: 13/540,887

(22) Filed: Jul. 3, 2012

Related U.S. Application Data
(60) Provisional application No. 61/505,006, filed on Jul. 6, 2011.

(51) Int. Cl. A61B 18/08; A61B 18/04; A61B 18/18

(52) U.S. Cl. CPC A61B 18/085; A61B 18/18; A61B 18/042
USPC 606/33; 606/45

(57) ABSTRACT

A surgical tissue cutting and extraction device includes a sleeve having a tissue extraction lumen. One or more jaw members are coupled to the sleeve and configured to pivot or flex relative to the sleeve to capture tissue. The captured tissue may then be resected using radio frequency or other cutting tools on the sleeve.

### CROSS-REFERENCE TO RELATED APPLICATIONS

This application claims the benefit of U.S. Provisional Application No. 61/505,006 (Attorney Docket No. 33291-717.101), filed on Jul. 6, 2011, the full disclosure of which is incorporated herein by reference.

### BACKGROUND OF THE INVENTION

1. Field of the Invention

The present invention relates to systems and methods for cutting and extracting tissue in endoscopic surgeries.

Electrosurgical cutting devices often comprise a shaft or sleeve having a tissue extraction lumen with one or more radio frequency (RF) cutting blades arranged to resect tissue which may then be drawn into the extraction lumen, often via vacuum assistance. Most such electrosurgical tissue cutting devices rely on manually engaging the electrode or other tissue-cutting edge against the target tissue to be resected. While such manual engagement is often sufficient, in other cases, such as in laparoscopic procedures having limited access, the target tissue can be difficult to immobilize prior to resection. For these reasons, it would be desirable to provide improved electrosurgical cutting tools having the ability to engage and immobilize tissue prior to cutting.

2. Description of the Background Art

Related patents and applications include U.S. Pat. No. 8,221,404; U.S. Pat. No.7,744,595; U.S. 2010/0305565; U.S. 2007/0213704; U.S. 2009/0270849; and U.S. Ser. No. 13/309,983.

### SUMMARY OF THE INVENTION

In general, a surgical tissue cutting device corresponding to the invention comprises (i) an axially-extending sleeve having a tissue extraction lumen wherein a distal end portion of the sleeve comprises an electrode edge, and (ii) one or more jaw members coupled to the sleeve wherein the jaw members are configured to pivot or flex exteriorly of the extraction lumen toward and away from one another. The jaw and sleeve are axially moveable relative to one another, and the electrode edge is coupled to an RF source and a controller for generating a tissue-cutting plasma at the electrode edge.

In one embodiment, the electrode edge comprises a first polarity electrode and at least one jaw comprises a second polarity electrode. The RF cutting sleeve can be actuatable axially and/or rotationally by a manual actuator, or a motor drive. Similarly, the jaws can be moveable toward or away from one another by a manual actuator or by a motor drive. A controller can actuate the jaws and the RF cutting sleeve drive in a selected sequence. In one variation, the system includes an RF on-off limit switch which terminates RF delivery based on the axial movement of the sleeve relative to the position of the jaws. For example, the RF on-off limit switch can be configured to terminate RF delivery when the electrode edge of the sleeve reached a predetermined extension distance relative to the distal end of the jaws. In another variation, the RF on-off limit switch can be configured to terminate RF delivery when an inner face of the jaws is within a predetermined proximity to the electrode edge of the sleeve.

As can be seen in FIGS. 7 and 8A, the electrode edge can define a plane that is transverse to said axis, or non-transverse relative to said axis.

In general, a surgical tissue cutting device comprises an outer sleeve and a concentric reciprocatable inner sleeve having a distal electrode edge configured for plasma formation to cut tissue, and first and second clamp elements extending from the outer sleeve for capturing and position tissue for cutting and extraction by said inner sleeve. The inner sleeve can define an interior lumen coupled to a negative pressure source for tissue extraction, wherein the interior lumen has a mean cross section of at least 4 mm, 6 mm or 8 mm. The clamp elements can define a zone therebetween and wherein movement of the electrode edge into the zone terminates energy delivery to said electrode edge. In another variation, the clamp elements can be configured in a surface region providing for non-contact with the cutting sleeve's electrode edge in any stage of reciprocation of the cutting sleeve and any relative position of said clamp elements.

In another embodiment, an electrosurgical tissue resection device comprises a shaft having a working end comprising first and second clamp elements, and at least one clamp element comprising an outer sleeve and a concentric reciprocatable inner sleeve having a distal electrode edge configured for plasma formation to resect tissue.

In another embodiment, an electrosurgical tissue resection device comprises a shaft extending to a working end comprising first and second clamp elements, at least one clamp element having a plurality of rotational points to allow the clamp elements to move toward one another in parallel or non-parallel relationships, and an RF electrode carried by the working end for resecting tissue.

In another embodiment, an electrosurgical tissue resection device comprises a shaft extending to a working end comprising first and second clamp elements, at least one clamp element comprising a spring-wire form capable of a first constrained sectional dimension and a second non-constrained dimension for capturing a tissue mass, and an RF electrode carried by the working end for resecting tissue.

In general, a method of the invention for removing targeted tissue from the interior of a patient's body comprises clamping tissue between first and second jaw members carried by a probe working end, energizing an RF electrode at a tissue-receiving opening of the probe to electrosurgically cut tissue, and extracting cut tissue through a tissue extraction passageway in the probe.

### BRIEF DESCRIPTION OF DRAWINGS

FIG. 1A is a perspective view of the working end of the tissue-cutting device with a reciprocating RF cutting sleeve in a non-extended position.

FIG. 1B is a perspective view of the tissue-cutting device of FIG. 1A with the reciprocating RF cutting sleeve in a partially extended position.

FIG. 1C is a perspective view of the tissue-cutting device of FIG. 1A with the reciprocating RF cutting sleeve in a fully extended position across the tissue-receiving window.

FIG. 2A is a sectional view of the working end of the tissue-cutting device of FIG. 1A with the reciprocating RF cutting sleeve in a non-extended position.

FIG. 2B is a sectional view of the working end of FIG. 1B with the reciprocating RF cutting sleeve in a partially extended position.

FIG. 2C is a sectional view of the working end of FIG. 1C with the reciprocating RF cutting sleeve in a fully extended position.

FIG. 3A is an enlarged sectional view of the working end of tissue-cutting device of FIG. 2B with the reciprocating RF cutting sleeve in a partially extended position showing the RF field in a first RF mode and plasma cutting of tissue.

FIG. 3B is an enlarged sectional view of the working end of FIG. 2C with the reciprocating RF cutting sleeve almost fully extended and showing the RF fields switching to a second RF mode from a first RF mode shown in FIG. 3A.

FIG. 3C is an enlarged sectional view of the working end of FIG. 2C with the reciprocating RF cutting sleeve again almost fully extended and showing the explosive vaporization of a captured liquid volume to expel cut tissue in the proximal direction.

FIG. 4 is an enlarged perspective view of another working end with an RF cutting sleeve and a clamp member.

FIG. 5 is another variation of working end with an RF cutting sleeve and tissue-clamp mechanism.

FIG. 6 is another variation of working end with an RF cutting sleeve and tissue-clamp mechanism.

FIG. 7 is another variation of working end with an RF cutting sleeve and tissue-clamp mechanism.

FIG. 8A is another variation of working end with an RF cutting sleeve and tissue-clamp mechanism in a first position.

FIG. 8B is another variation of working end with an RF cutting sleeve and tissue-clamp mechanism in a second position.

FIG. 9 is a perspective view of a helically wound electrode for a cutting sleeve as in FIG. 6.

FIG. 10 is a perspective view of a variation of a helically wound electrode for an RF cutting sleeve.

FIG. 11 is an illustration of another variation of a helically wound electrode for an RF cutting sleeve.

FIG. 12 is an illustration of another variation of a helically wound electrode for an RF cutting sleeve.

### DETAILED DESCRIPTION

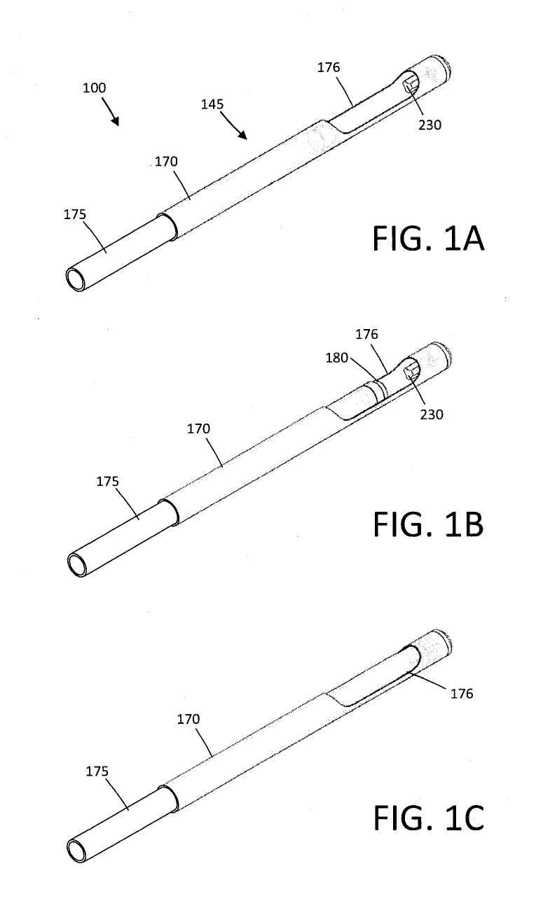

FIGS. 1A-1C illustrate a working end 145 of a tissue-cutting device 100 with an elongated windowed outer sleeve 170 and inner cutting sleeve configured to extend across window. A handle of the tissue-cutting device 100 is adapted for manipulating the electrosurgical working end 145 of the device. The tissue-cutting device 100 has subsystems coupled to its handle to enable electrosurgical cutting of targeted tissue. A radiofrequency generator or RF source 150 and controller 155 are coupled to at least one RF electrode carried by the working end 145 as will be described in detail below. In one embodiment shown in FIG. 1, an electrical cable 156 and negative pressure source 125 are operatively coupled to a connector 158 in handle 142. The electrical cable couples the RF source 150 to the electrosurgical working end 145. The negative pressure source 125 communicates with a tissue-extraction channel 160 in the shaft assembly 140 of the tissue extraction device 100.

In one embodiment, the handle 142 of the tissue-cutting device 100 includes a motor drive 165 for reciprocating or otherwise moving a cutting component of the electrosurgical working end 145. The handle optionally includes one or more actuator buttons for actuating the device. In another embodiment, a footswitch can be used to operate the device. In one embodiment, the system includes a switch or control mechanism to provide a plurality of reciprocation speeds, for example 1 Hz, 2 Hz , 3 Hz, 4 Hz and up to 8 Hz. Further, the system can include a mechanism for moving and locking the reciprocating cutting sleeve in a non-extended position and in an extended position. Further, the system can include a mechanism for actuating a single reciprocating stroke.

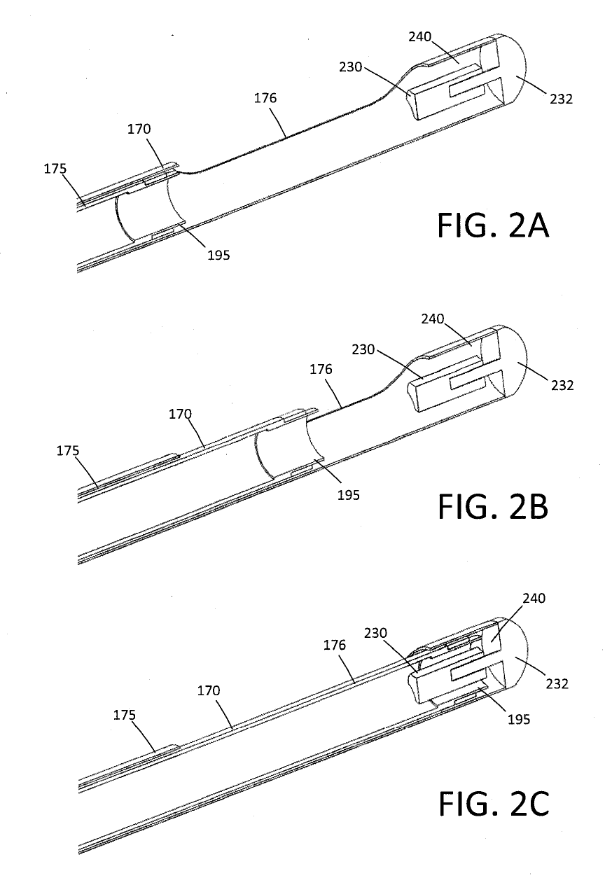

Referring to FIGS. 1A-2C, one variation of electrosurgical tissue-cutting device has an elongate shaft assembly extending about longitudinal axis comprising an exterior or first outer sleeve 170 with passageway or lumen 172 therein that accommodates a second or inner sleeve 175 that can reciprocate (and optionally rotate or oscillate) in lumen 172 to cut tissue as is known in that art of such tubular cutters. In one embodiment, the tissue-receiving window 176 in the outer sleeve 170 has an axial length ranging between 10 mm and 30 mm and extends in a radial angle about outer sleeve 170 from about 45° to 210° relative to axis 168 of the sleeve. The outer and inner sleeves 170 and 175 can comprise a thin-wall stainless steel material and function as opposing polarity electrodes as will be described in detail below. Insulative layers are carried by the outer and inner sleeves 170 and 175 to limits, control and/or prevent unwanted electrical current flows between certain portions go the sleeve. In one embodiment, a stainless steel outer sleeve 170 has an O.D. of 0.143″ with an I.D. of 0.133″ and with an inner insulative layer (described below) the sleeve has a nominal I.D. of 0.125″. In this embodiment, the stainless steel inner sleeve 175 has an O.D. of 0.120″ with an I.D. of 0.112″. The inner sleeve 175 with an outer insulative layer has a nominal O.D. of about 0.123″ to 0.124″ to reciprocate in lumen 172. In other embodiments, outer and or inner sleeves can be fabricated of metal, plastic, ceramic of a combination thereof. The cross-section of the sleeves can be round, oval or any other suitable shape.

In one embodiment, the distal end 177 of inner sleeve 175 comprises a first polarity electrode with distal cutting electrode edge 180 about which plasma can be generated. The electrode edge 180 also can be described as an active electrode during tissue cutting since the electrode edge 180 then has a substantially smaller surface area than the opposing polarity or return electrode. In one embodiment, the exposed surfaces of outer sleeve 170 comprises the second polarity electrode 185, which thus can be described as the return electrode since during use such an electrode surface has a substantially larger surface area compared to the functionally exposed surface area of the active electrode edge 180.

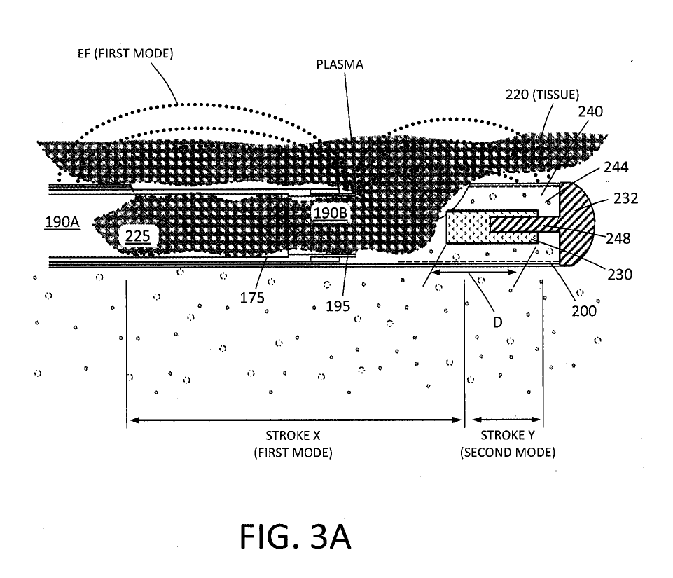

In one aspect of the invention, the inner sleeve or cutting sleeve 175 has an interior tissue extraction lumen 160 with first and second interior diameters that are adapted to electrosurgically cut tissue volumes rapidly—and thereafter consistently extract the cut tissue strips through the highly elongated lumen 160 without clogging. In one variation, the inner sleeve 175 has a first diameter portion 190A that extends from a handle to a distal region 192 of the sleeve 175 wherein the tissue extraction lumen transitions to a smaller second diameter lumen 190B with a reduced diameter indicated at B which is defined by the electrode sleeve element 195 that provides cutting electrode edge 180. The axial length C of the reduced cross-section lumen 190B can range from about 2 mm to 20 mm. In one embodiment, the first diameter A is 0.112″ and the second reduced diameter B is 0.100″. As shown in FIG. 3A, the inner sleeve 175 can be an electrically conductive stainless steel and the reduced diameter electrode portion also can comprise a stainless steel electrode sleeve element 195 that is welded in place by weld 196. In another alternative embodiment, the electrode and reduced diameter electrode sleeve element 195 comprises a tungsten tube that can be press fit into the distal end 198 of inner sleeve 175. In one variation, the outer sleeve 170 is lined with a thin-wall insulative material 200, such as PFA, or another material described below. Similarly, the inner sleeve 175 has an exterior insulative layer 202. These coating materials can be lubricious as well as electrically insulative to reduce friction during reciprocation of the inner sleeve 175.

The insulative layers 200 and 202 described above can comprise a lubricious, hydrophobic or hydrophilic polymeric material. For example, the material can comprise a bio-compatible material such as PFA, TEFLON®, polytetrafluroethylene (PTFE), FEP (Fluorinated ethylenepropylene), polyethylene, polyamide, ECTFE (Ethylenechlorotrifluoro-ethylene), ETFE, PVDF, polyvinyl chloride or silicone.

Now turning to FIG. 3A, one variation of inner sleeve 175 is illustrated in a schematic view together with a tissue volume being resected with the plasma electrode edge 180. In this embodiment, as in other embodiments in this disclosure, the RF source operates at selected operational parameters to create a plasma around the electrode edge 180 of electrode sleeve 195 as is known in the art. Thus, the plasma generated at electrode edge 180 can cut and ablate a path in the tissue 220, and is suited for cutting any targeted tissue. In FIG. 3A, the distal portion of the cutting sleeve 175 includes a ceramic collar 222 which is adjacent the distal edge 180 of the electrode sleeve 195. The ceramic 222 collar functions to confine plasma formation about the distal electrode edge 180 and functions further to prevent plasma from contacting and damaging the polymer insulative layer 202 on the cutting sleeve 175 during operation. In one aspect of the invention, the path P cut in the tissue 220 with the plasma at electrode edge 180 provides a path P having an ablated width indicated at W, wherein such path width W is substantially wide due to tissue vaporization. This removal and vaporization of tissue in path P is substantially different than the effect of cutting similar tissue with a sharp blade edge, as in various prior art devices. A sharp blade edge can divide tissue (without cauterization) but applies mechanical force to the tissue and may prevent a large cross section slug of tissue from being cut. In contrast, the plasma at the electrode edge 180 can vaporize a path P in tissue without applying any substantial force on the tissue to thus cut larger cross sections or slugs or strips of tissue. Further, the plasma cutting effect reduces the cross section of tissue strip 225 received in the tissue-extraction lumen 190B. FIGS. 3A-3B depicts a tissue strip 225 entering lumen 190B which has such a smaller cross-section than the lumen due to the vaporization of tissue. Further, the cross section of tissue 225 as it enters the larger cross-section lumen 190A results in even greater free space 196 around the tissue strip 225. Thus, the resection of tissue with the plasma electrode edge 180, together with the lumen transition from the smaller cross-section (190B) to the larger cross-section (190A) of the tissue-extraction lumen 160 can significantly reduce or eliminate the potential for successive resected tissue strips 225 to clog the lumen. Prior art resection devices with such small diameter tissue-extraction lumen typically have problems with tissue clogging.

In general, one aspect of the invention comprises a tissue cutting and extracting device (FIGS. 1A-3C) that includes first and second concentric sleeves having an axis and wherein the second (inner) sleeve 175 has an axially-extending tissue-extraction lumen therein, and wherein the second sleeve 175 is moveable between axially non-extended and extended positions relative to a tissue-receiving window 176 in first sleeve 170 to resect tissue, and wherein the tissue extraction lumen 160 has first and second cross-sections. The second sleeve 175 has a distal end configured as a plasma electrode edge 180 to resect tissue disposed in tissue-receiving window 176 of the first sleeve 170. Further, the distal end of the second sleeve, and more particularly, the electrode edge 180 is configured for plasma ablation of a substantially wide path in the tissue. In general, the tissue-extraction device is configured with a tissue extraction lumen 160 having a distal end portion with a reduced cross-section that is smaller than a cross-section of medial and proximal portions of the lumen 160.

FIGS. 1A-3C illustrate the working end 145 of the tissue-cutting device 100 with the reciprocating cutting sleeve or inner sleeve 175 in different axial positions relative to the tissue receiving window 176 in outer sleeve 170. In FIG. 1A, the cutting sleeve 175 is shown in a retracted or non-extended position in which the sleeve 175 is at it proximal limit of motion and is prepared to advance distally to an extended position to thereby electrosurgically cut tissue positioned in and/or suctioned into in window 176. FIG. 1B shows the cutting sleeve 175 moved and advanced distally to a partially advanced or medial position relative to tissue cutting window 176. FIG. 1C illustrates the cutting sleeve 175 fully advanced and extended to the distal limit of its motion wherein the plasma cutting electrode 180 has extended past the distal end 226 of tissue-receiving window 176 at which moment the resected tissue strip 225 in excised from tissue volume 220 and captured in reduced cross-sectional lumen region 190A.

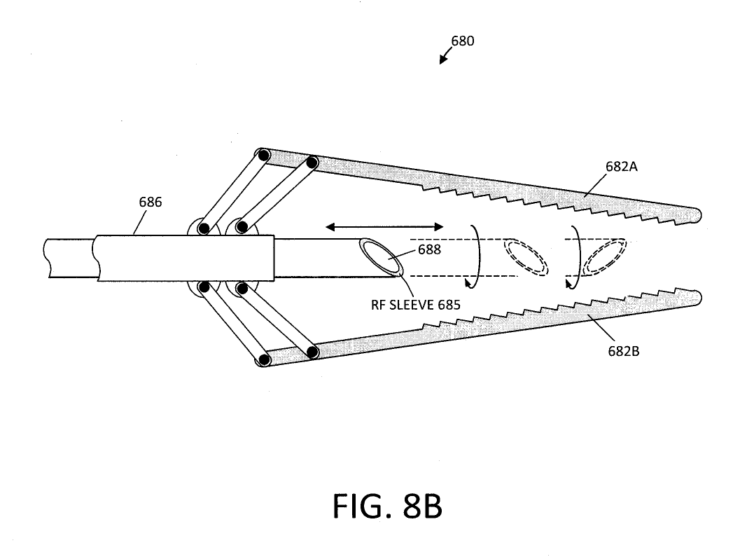

Now referring to FIGS. 2A-2C and FIGS. 3A-3C, another aspect of the invention comprises “tissue displacement” mechanisms provided by multiple elements and processes to “displace” and move tissue strips 225 in the proximal direction in lumen 160 of cutting sleeve 175 to thus ensure that tissue does not clog the lumen of the inner sleeve 175. As can seen in FIG. 10A and the enlarged views of FIGS. 2A-2, one tissue displacement mechanism comprises a projecting element 230 that extends proximally from distal tip 232 which is fixedly attached to outer sleeve 170. The projecting element 230 extends proximally along central axis 168 in a distal chamber 240 defined by outer sleeve 170 and distal tip 232. In one embodiment depicted in FIG. 2A, the shaft-like projecting element 230, in a first functional aspect, comprises a mechanical pusher that functions to push a captured tissue strip 225 proximally from the small cross-section lumen 190B of cutting sleeve 175 as the cutting sleeve 175 moves to its fully advanced or extended position. In a second functional aspect, the chamber 240 in the distal end of sleeve 170 is configured to capture a volume of saline distending fluid 244 from the working space, and wherein the existing RF electrodes of the working end 145 are further configured to explosively vaporize the captured fluid 244 to generate proximally-directed forces on tissue strips 225 resected and disposed in lumen 160 of the cutting sleeve 175. Both of these two functional elements and processes (tissue displacement mechanisms) can apply a substantial mechanical force on the captured tissue strips 225 by means of the explosive vaporization of liquid in chamber 240 and can function to move tissue strips 225 in the proximal direction in the tissue-extraction lumen 160. It has been found that using the combination of multiple functional elements and processes can virtually eliminate the potential for tissue clogging the tissue extraction lumen 160.

More in particular, FIGS. 3A-3C illustrate sequentially the functional aspects of the tissue displacement mechanisms and the explosive vaporization of fluid captured in chamber 240. In FIG. 3A, the reciprocating cutting sleeve 175 is shown in a medial position advancing distally wherein plasma at the cutting electrode edge 180 is cutting a tissue strip 225 that is disposed within lumen 160 of the cutting sleeve 175. In FIGS. 3A-3C, it can be seen that the system operates in first and second electrosurgical modes corresponding to the reciprocation and axial range of motion of cutting sleeve 175 relative to the tissue-receiving window 176. As used herein, the term “electrosurgical mode” refers to which electrode of the two opposing polarity electrodes functions as an “active electrode” and which electrode functions as a “return electrode”. The terms “active electrode” and “return electrode” are used in accordance with convention in the art—wherein an active electrode has a smaller surface area than the return electrode which thus focuses RF energy density about such an active electrode. In the working end 145 of FIGS. 2A-2C, the cutting electrode element 195 and its cutting electrode edge 180 must comprise the active electrode to focus energy about the electrode to generate the plasma for tissue cutting. Such a high-intensity, energetic plasma at the electrode edge 180 is needed throughout stroke X indicated in FIG. 3A-3B to cut tissue. The first mode occurs over an axial length of travel of inner cutting sleeve 175 as it crosses the tissue-receiving window 176, at which time the entire exterior surface of outer sleeve 170 comprises the return electrode indicated at 185. The electrical fields EF of the first RF mode are indicated generally in FIG. 3A.

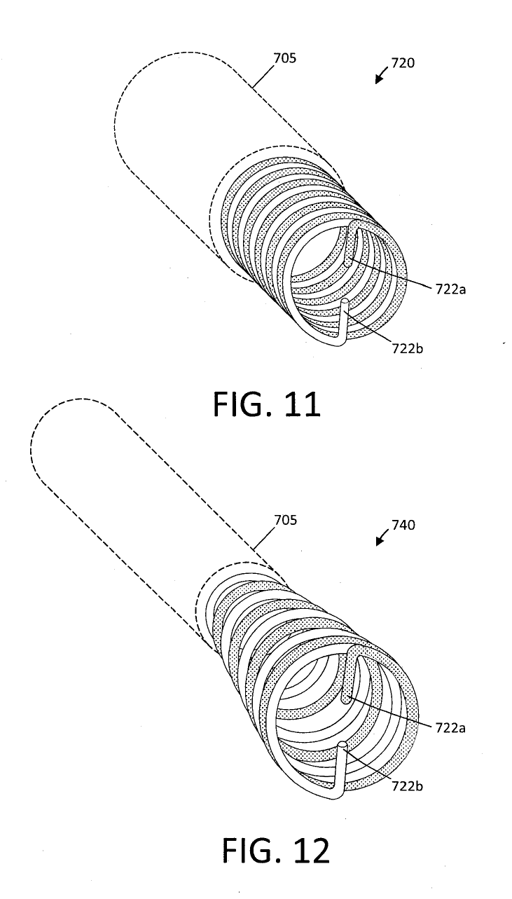

FIG. 3B illustrates the moment in time at which the distal advancement or extension of inner cutting sleeve 175 entirely crossed the tissue-receiving window 176. At this time, the electrode sleeve 195 and its electrode edge 180 are confined within the mostly insulated-wall chamber 240 defined by the outer sleeve 170 and distal tip 232. At this moment, the system is configured to switch to the second RF mode in which the electric fields EF switch from those described previously in the first RF mode. As can be seen in FIG. 12B, in this second mode, the limited interior surface area 250 of distal tip 232 that interfaces chamber 240 functions as an active electrode and the distal end portion of cutting sleeve 175 exposed to chamber 240 acts as a return electrode. In this mode, very high energy densities occur about surface 250 and such a contained electric field EF can explosively and instantly vaporize the fluid 244 captured in chamber 240. The expansion of water vapor can be dramatic and can thus apply tremendous mechanical forces and fluid pressure on the tissue strip 225 to move the tissue strip in the proximal direction in the tissue extraction lumen 160. FIG. 3C illustrates such explosive or expansive vaporization of the distention fluid 244 captured in chamber 240 and further shows the tissue strip 225 being expelled in the proximal direction the lumen 160 of inner cutting sleeve 175. FIG. 14 further shows the relative surface areas of the active and return electrodes at the extended range of motion of the cutting sleeve 175, again illustrating that the surface area of the non-insulated distal end surface 250 is small compared to surface 255 of electrode sleeve which comprises the return electrode.

Still referring to FIGS. 3A-3C, it has been found that a single power setting on the RF source 150 and controller 155 can be configured both (i) to create plasma at the electrode cutting edge 180 of electrode sleeve 195 to cut tissue in the first mode, and (ii) to explosively vaporize the captured distention fluid 244 in the second mode. Further, it has been found that the system can function with RF mode-switching automatically at suitable reciprocation rates ranging from 0.5 cycles per second to 8 or 10 cycles per second. In bench testing, it has been found that the tissue-cutting device described above can cut and extract tissue at the rate of from 4 grams/min to 8 grams/min without any potential for tissue strips 225 clogging the tissue-extraction lumen 160. In these embodiments, the negative pressure source 125 also is coupled to the tissue-extraction lumen 160 to assist in applying forces for tissue extraction.

Of particular interest, the fluid-capture chamber 240 defined by sleeve 170 and distal tip 232 can be designed to have a selected volume, exposed electrode surface area, length and geometry to optimize the application of expelling forces to resected tissue strips 225. In one embodiment, the diameter of the chamber is 3.175 mm and the length is 5.0 mm which taking into account the projecting element 230, provided a captured fluid volume of approximately 0.040 mL. In other variations, the captured fluid volume can range from 0.004 to 0.080 mL.

In one example, a chamber 240 with a captured liquid volume of 0.040 mL together with 100% conversion efficiency in and instantaneous vaporization would require 103 Joules to heat the liquid from room temperature to water vapor. In operation, since a Joule is a W*s, and the system reciprocate at 3 Hz, the power required would be on the order of 311 W for full, instantaneous conversion to water vapor. A corresponding theoretical expansion of 1700× would occur in the phase transition, which would results in up to 25,000 psi instantaneously (14.7 psi×1700), although due to losses in efficiency and non-instantaneous expansion, the actual pressures would be much less. In any event, the pressures are substantial and can apply significant expelling forces to the captured tissue strips 225.

Referring to FIG. 3A, the interior chamber 240 can have an axial length from about 0.5 mm to 10 mm to capture a liquid volume ranging from about 0.004 mL 0.01 mL. It can be understood in FIG. 12A, that the interior wall of chamber 240 has an insulator layer 200 which thus limits the electrode surface area 250 exposed to chamber 240. In one embodiment, the distal tip 232 is stainless steel and is welded to outer sleeve 170. The post element 248 is welded to tip 232 or machined as a feature thereof. The projecting element 230 in this embodiment is a non-conductive ceramic.

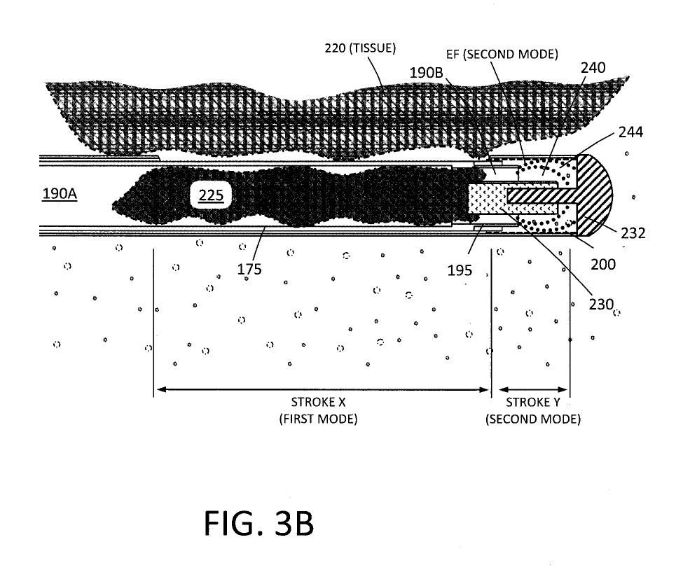

FIG. 4 illustrates a working end 600 of a tissue-cutting device adapted for laparoscopic tissue morcellation, for example, to cut and remove tissue from an CO₂ insufflated working space. The working end has jaw portions 605 and 610 with jaw 610 being moveable to capture tissue and push tissue into a reciprocating RF cutting sleeve 620 similar to the RF cutting sleeve 170 described above. The actuatable jaw 610 can be moved by any mechanism, such as extendable sleeve 622 that engages cam surfaces 624 of jaw 610. The jaw portion 605 that carries the cutting sleeve can 620 an have any round, oval or rectangular cross section. In one embodiment, the actuatable jaw 610 comprises a wire frame that is resilient and can expand laterally (phantom view) after insertion into a body space wherein such an expanded jaw can engage a larger surface area of an organ or tissue volume targeted for resection.

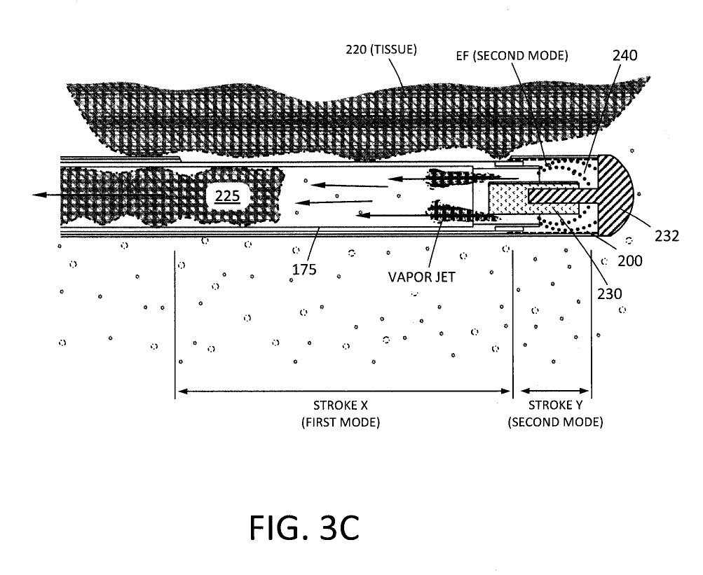

FIG. 5 illustrates another working end 630 which is configured with a jaw-closing mechanism in which a jaw or clamp member 632A can close in a parallel manner with opposing jaw or clamp member 632B which is again configured with the RF cutting sleeve and tissue extraction channel.

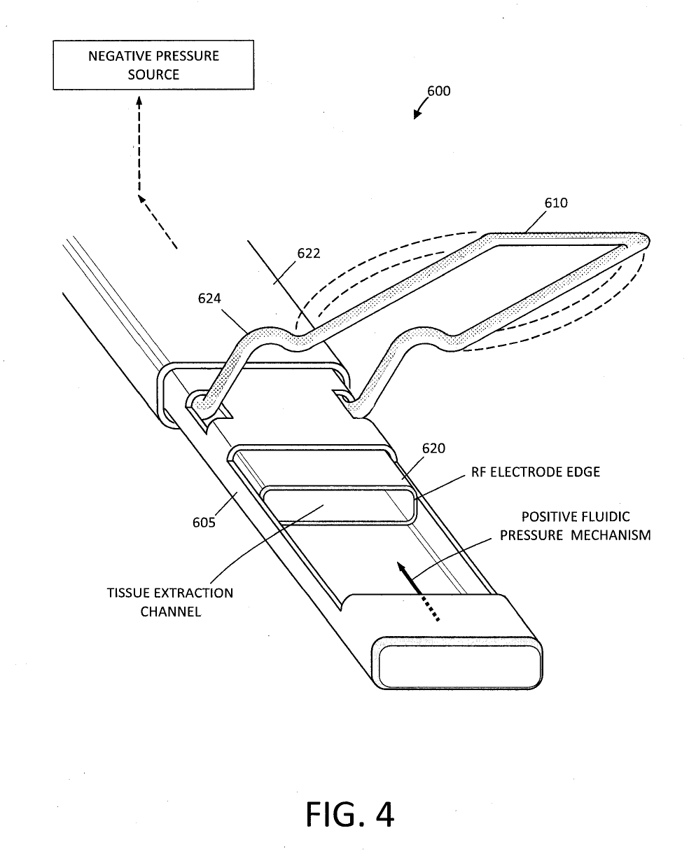

FIG. 6 schematically illustrates working end 640 with jaws or clamp members 642A and 642B wherein both clamp members 642A, 642B are configured with RF cutting sleeves and tissue extraction channels. The tissue extraction channels can merge into a single channel in shaft 645. In any of the embodiments of FIGS. 4-6, the systems can include a positive fluidic pressure source in communication with the distal end of the extraction channel as described above, or alternatively a high pressure flow of a gas or liquid from a remote source that flows through a lumen in the sleeve assembly and wherein the gas or liquid is then jetted proximally from an outlet in the interior of the working end. In one embodiment, the remote source comprises a pressurized CO₂ canister.

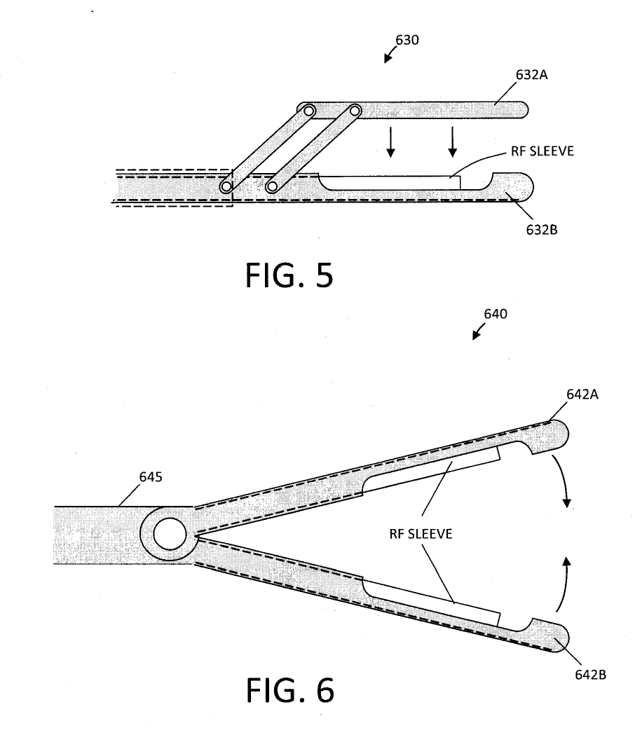

FIG. 7 illustrates another working end 650 which is configured with a central RF cutting sleeve 655 and tissue extraction channel therein. The clamp members 652A and 652B are carried by an actuatable in an assembly outward of the extraction channel. This embodiment allows for the maximum diameter cross section of the extraction channel for rapid tissue cutting and extraction. The RF cutting sleeve is then reciprocated and/or rotated in the tissue volume captured by the clamp members.

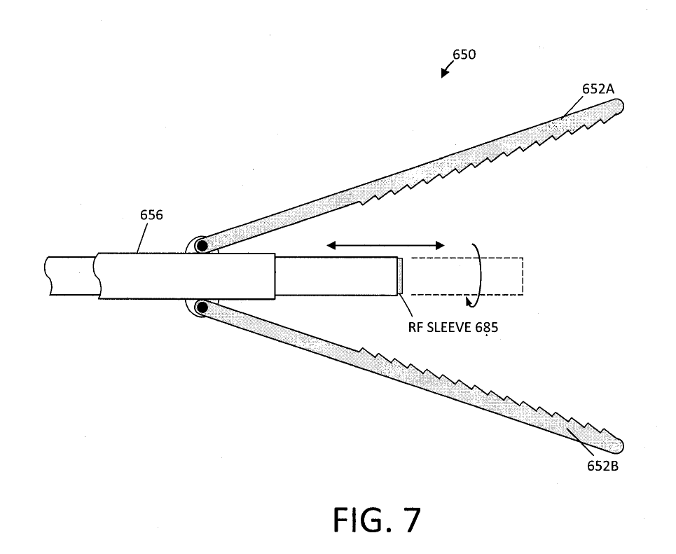

FIGS. 8A-8B illustrate another working end 680 which is configured with a multi-pivot multi-link jaw-closing mechanism in which the clamp elements 682A, 682B can close in a parallel or non-parallel manner to feed tissue into the central RF cutting sleeve 685—which again is actuatable relative to housing sleeve 686 and the clamp elements. A negative pressure source is coupled to the tissue extraction channel 688 in the RF cutting sleeve 685 as described previously. FIG. 8B depicts the multi-link actuator moving the clamps inwardly in a non-parallel manner to feed tissue to the cutting sleeve.

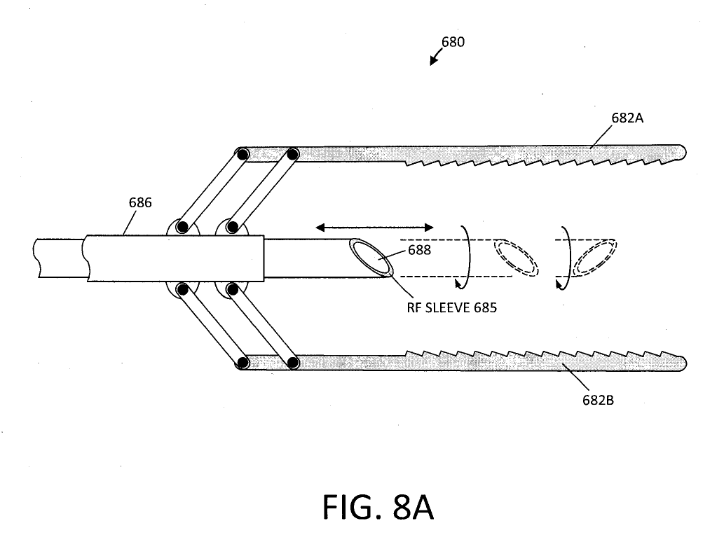

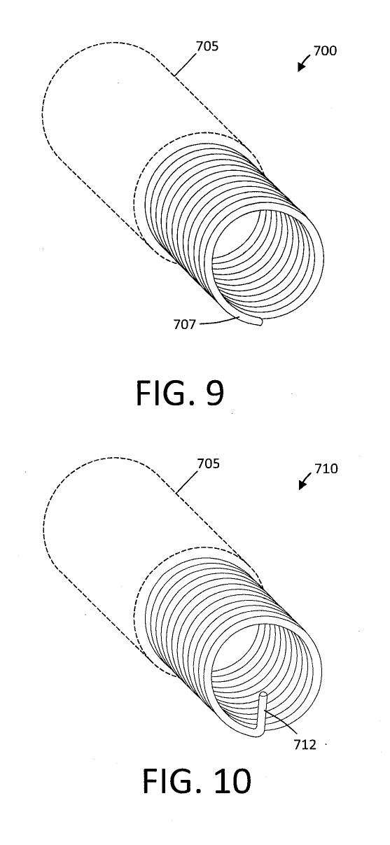

FIG. 9 illustrates a distal working end 700 of a RF cutting sleeve wherein sleeve 705 is coupled to a helical element comprising stainless steel or tungsten wire 707 or the like that comprises the electrode about which a cutting plasma is formed. The helical winding allows for a thin cross-sections wall (i.e., wire diameter). This electrode 707 can be used in the embodiment shown in FIGS. 7-8B. FIG. 10 shows another embodiment 710 in which a wire end 712 extends inwardly to thereby cut tissue in a different form with a potentially smaller cross section for more rapid and efficient extraction through the interior lumen. FIG. 11 shows another embodiment 720 in which two wires are helically intertwined to provide two ends 722a, 722b that extend inwardly to cut tissue longitudinally in each extracted tissue strip. FIG. 12 shows another embodiment 740 in which two wires are again helically intertwined to provide two ends 722a, 722b that extend inwardly to cut tissue longitudinally to reduce the tissue cross section. In this embodiment, the wires are resilient and can move from a constrained cross section to an expanded cross section to more rapidly cut and extract tissue. Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

What is claimed is:

1. A surgical tissue cutting and extraction device, comprising:
an axially-extending sleeve having a tissue extraction lumen;
an electrode edge disposed at a distal end of the sleeve; and
one or more jaw members coupled to the sleeve, the jaw members configured to pivot or flex exteriorly of the extraction lumen toward and away from one another to capture tissue therebetween.

2. The surgical device of claim 1 wherein at least one jaw and sleeve are axially moveable relative to one another.

3. The surgical device of claim 1 wherein the electrode edge is coupled to an RF source and a controller for generating a tissue-cutting plasma at the electrode edge.

4. The surgical device of claim 1 wherein the electrode edge comprises a first polarity electrode and at least one jaw comprises a second polarity electrode.

5. The surgical device of claim 1 wherein the sleeve is axially moveable relative to the jaw member.

6. The surgical device of claim 1 wherein the sleeve is rotationally moveable relative to the jaw member.

7. The surgical device of claim 1 wherein the sleeve is actuatable axially and/or rotationally by a manual actuator.

8. The surgical device of claim 1 further comprising a motor drive to axially and/or rotationally actuate the sleeve.

9. The surgical device of claim 1 wherein the jaws are moveable toward or away from one another by a manual actuator.

10. The surgical device of claim 1 further comprising a motor drive to move the jaws are moveable toward or away from one another by a motor drive.

11. The surgical device of claim 1 wherein the jaws and sleeve are actuatable by a motor drive in a selected sequence.

12. The surgical device of claim 1 further comprising an RF on-off limit switch which terminates RF delivery based on the axial movement of the sleeve relative to the position of the jaws.

13. The surgical device of claim 12 wherein the RF on-off limit switch is configured to terminate RF delivery when the electrode edge of the sleeve reached a predetermined extension distance relative to the distal end of the jaws.

14. The surgical device of claim 12 wherein the RF on-off limit switch is configured to terminate RF delivery when an inner face of the jaws is within a predetermined proximity to the electrode edge of the sleeve.

15. The surgical device of claim 1 wherein the electrode edge defines a plane that is transverse to said axis.

16. The surgical device of claim 1 wherein the electrode edge defines a plane that is non-transverse relative to said axis.

17. The surgical device of claim 1 wherein the electrode edge defines a plane that is non-transverse relative to said axis.

18. The surgical device of claim 1 wherein the electrode edge defines a plane that is angled relative to said axis.

19. The surgical device of claim 1 wherein the electrode edge defines a window that is substantially parallel to said axis.

20. The surgical device of claim 1 further comprising a negative pressure source in communication with a proximal end of the extraction channel.

21. The surgical device of claim 1 further comprising a source of positive fluidic pressure in communication with a distal end portion of the extraction channel.

22. The surgical device of claim 21 wherein the source of positive fluidic pressure comprises an outlet in fluid communication with remote pressurized liquid source.

23. The surgical device of claim 21 wherein the source of positive fluidic pressure comprises an outlet in fluid communication with remote pressurized gas source.

24. The surgical device of claim 21 wherein the source of positive fluidic pressure comprises a liquid source in communication with a distal chamber having an electrode arrangement for explosive vaporization of the said liquid to apply said fluidic pressure.

25. A surgical tissue cutting and extraction device, comprising:
an outer sleeve and a concentric reciprocatable inner sleeve having a distal electrode edge configured for plasma formation to cut tissue; and
first and second clamp elements extending from the outer sleeve for capturing and position tissue for cutting and extraction by said inner sleeve.

26. The surgical device of claim 25 wherein the inner sleeve defines an interior lumen coupled to a negative pressure source for tissue extraction.

27. The surgical device of claim 25 wherein the interior lumen has a mean cross section of at least 4 mm, 6 mm or 8 mm.

28. The surgical device of claim 25 wherein the first clamp element is actuatable relative to the outer sleeve to move toward the second clamp element.

29. The surgical device of claim 25 wherein the second clamp element is fixed relative to the outer sleeve.

30. The surgical device of claim 25 wherein the first and second clamp elements are actuatable relative to the outer sleeve to move toward one another.

31. The surgical device of claim 25 wherein the inner sleeve is axially and rotationally moveable relative to a clamp element.

32. The surgical device of claim 25 wherein the inner sleeve is reciprocatable and/or rotatable by manual and/or motor actuation.

33. The surgical device of claim 25 wherein the clamp elements are moveable relative to one another by a manual and/or motor actuation.

34. The surgical device of claim 25 wherein the electrode edge comprises a first polarity electrode and at least one clamp element comprises a second polarity electrode.

35. The surgical device of claim 25 wherein the clamp elements define a zone therebetween and wherein movement of the electrode edge into the zone terminates energy delivery to said electrode edge.

36. The surgical device of claim 34 wherein the clamp elements comprise said second polarity electrodes in a surface region configured for non-contact with said electrode edge in any stage of reciprocation of said inner sleeve and any relative position of said clamp elements.

37. The surgical device of claim 26 further comprising a source of positive fluidic pressure in communication with a distal end portion of the interior lumen.

38. An electrosurgical tissue resection device, comprising:
a shaft having a working end comprising first and second clamp elements; and
at least one clamp element comprising an outer sleeve and a concentric reciprocatable inner sleeve having a distal electrode edge configured for plasma formation to resect tissue.

39. The electrosurgical device of claim 38 wherein inner sleeve defines a tissue-extraction lumen.

40. The electrosurgical device of claim 38 wherein the first clamp element is moveable relative to the shaft.

41. The electrosurgical device of claim 38 wherein the second clamp element is fixed relative to the shaft.

42. The electrosurgical device of claim 38 wherein both the first and second clamp elements are moveable relative to the shaft.

43. The electrosurgical device of claim 39 further comprising a negative pressure source in communication with the tissue-extraction lumen.

44. The surgical device of claim 39 further comprising a source of positive fluidic pressure in communication with a distal end portion of the tissue-extraction lumen.

45. An electrosurgical tissue resection device, comprising:
a shaft extending to a working end comprising first and second clamp elements;
at least one clamp element having a plurality of rotational points to allow the clamp elements to move toward one another in parallel or non-parallel relationships; and
an RF electrode carried by the working end for resecting tissue.

46. The electrosurgical device of claim 45 wherein the RF electrode is disposed about an opening to tissue-extraction lumen extending from said working end to a proximal end of the shaft.

47. The electrosurgical device of claim 45 wherein the RF electrode is moveable axially and/or rotatably relative to a clamp element.

48. An electrosurgical tissue resection device, comprising:
a shaft extending to a working end comprising first and second clamp elements;
at least one clamp element comprising a spring-wire form capable of a first constrained sectional dimension and a second non-constrained dimension for capturing a tissue mass; and
an RF electrode carried by the working end for resecting tissue.

49. The electrosurgical device of claim 45 further comprising a tissue-extraction lumen extending from said working end to a proximal end of the shaft.

50. A surgical tissue cutting device, comprising:
an axially-extending sleeve having a tissue extraction lumen, a distal end portion of the sleeve comprising an electrode;
wherein the electrode comprises at least one helical electrode element.

51. The surgical tissue device of claim 50 wherein the helical electrode element comprises stainless steel or tungsten.

52. The surgical tissue device of claim 50 wherein the helical electrode element has a first constrained sectional dimension and a second non-constrained sectional dimension.

53. The surgical tissue device of claim 50 wherein the electrode comprises a plurality of intertwined helical electrode elements.

54. The surgical tissue device of claim 50 wherein a helical electrode element is configured with a terminal portion that extends non-circumferentially.

55. The surgical tissue device of claim 50 wherein a helical electrode element is configured with a terminal portion that extends inwardly toward the axis of the sleeve.

56. The surgical tissue device of claim 53 wherein a plurality of helical electrode element are configured with terminal portions that extends inwardly toward the axis of the sleeve.

57. A method of removing targeted tissue from the interior of a patient's body, comprising:
clamping tissue between first and second jaw members carried by a probe working end;
energizing an RF electrode at a tissue-receiving opening of the probe to electrosurgically cut tissue; and
extracting cut tissue through a tissue extraction passageway in the probe.

58. A method of removing targeted tissue from the interior of a patient's body, comprising:
clamping tissue between first and second jaw members carried by a probe working end;
energizing an RF electrode proximate the tissue-receiving structure on a jaw member to electrosurgically cut tissue; and
removing cut tissue through a tissue extraction passageway in jaw member.

59. The method of claim 58 wherein both the first and second jaw members carry RF electrodes.

60. The method of claim 58 wherein both the first and second jaw members are configured with tissue-receiving structures and tissue extraction passageways.
